(19) United States
(12) Patent Application Publication
(10) Pub. No.: US 2020/0294234 A1
(43) Pub. Date: Sep. 17, 2020

(54) METHODS AND SYSTEMS FOR AUTOMATEDLY COLLECTING AND RANKING DERMATOLOGICAL IMAGES

(71) Applicant: Matchlab, Inc., Cambridge, MA (US)

(72) Inventors: Nicholas Rance, Cambridge, MA (US); Alexander Taguchi, Cambridge, MA (US); Divya Sharma, Providence, RI (US); Nikki Riser, Cambridge, MA (US)

(21) Appl. No.: 16/818,629

(22) Filed: Mar. 13, 2020

Related U.S. Application Data
(60) Provisional application No. 62/817,653, filed on Mar. 13, 2019.

(51) Int. Cl. G06T 7/00; H04N 5/232; G06K 9/62; G06K 9/03; A61B 5/00

(52) U.S. Cl. CPC G06T 7/0014; H04N 5/23203; G06K 9/6256; G06K 9/627; G06K 9/036; G06K 2209/05; A61B 5/7275; A61B 5/445; G06T 2207/30168; G06T 2207/30088; G06T 2207/20081; A61B 5/0077

(57) ABSTRACT

In an aspect, a system for automatedly ranking dermatological images includes an image analysis device designed and configured to receive a plurality of images of a skin surface, detect, using a machine-learning process, an anatomical feature of interest in each image of the images of the skin surface, determine a degree of quality of depiction of the anatomical feature in each image of the plurality of images, and rank the plurality images according to degree of quality of depiction of the anatomical feature in each image.

### CROSS-REFERENCE TO RELATED APPLICATIONS

This application claims the benefit of priority of U.S. Provisional Patent Application Ser. No. 62/817,653, filed on Mar. 13, 2019, and titled “METHODS AND SYSTEMS FOR AUTOMATEDLY COLLECTING AND RANKING DERMATOLOGICAL IMAGES,” which is incorporated by reference herein in its entirety.

### FIELD OF THE INVENTION

The present invention generally relates to the field of computer vision and artificial intelligence. In particular, the present invention is directed to methods and systems for automatically collecting and ranking dermatological images.

### BACKGROUND

Diagnosis of conditions, questions about conditions, and tracking and evaluation of treatment with regard to dermatology are often facilitated by images taken of skin. For instance, a current or prospective patient of a dermatologist or other doctor may send an image of his or her skin to illustrate a symptom the current or prospective patient is experiencing. A person or group of people undergoing treatment for a given dermatological condition may have their progress tracked by a series of photographs of affected skin areas. People being trained to evaluated and treat skin conditions may also be trained to recognize such conditions using images. Unfortunately, many skin images in image banks or taken by users are of poor quality, either lacking in focal clarity or adequate light levels, or otherwise failing to depict features of interest reliably, leading to misdiagnoses or delays in communication. This problem is exacerbated where there is a lack of images for all demographic varieties of skin, or where people such as dermatologists are trained without regard to the diversity of skin types.

### SUMMARY OF THE DISCLOSURE

In an aspect, a system for automatedly ranking dermatological images includes an image analysis device designed and configured to receive a plurality of images of a skin surface, detect, using a machine-learning process, an anatomical feature of interest in each image of the images of the skin surface, determine a degree of quality of depiction of the anatomical feature in each image of the plurality of images, and rank the plurality images according to degree of quality of depiction of the anatomical feature in each image.

In another aspect a method of automatedly ranking dermatological images includes receiving, by a computing device, a plurality of images of a skin surface, detecting, by the computing device and using a machine-learning process, an anatomical feature of interest in each image of the images of the skin surface, determining, by the computing device, a degree of quality of depiction of the anatomical feature in each image of the plurality of images, and ranking, by the computing device, the plurality images according to degree of quality of depiction of the anatomical feature in each image.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for collecting and ranking dermatological images;

FIG. 2 is a block diagram illustrating an exemplary embodiment of an image database;

FIG. 3 is a flow diagram illustrating an exemplary embodiment of a method of automatedly evaluating dermatological images;

FIG. 4 is a flow diagram illustrating an exemplary embodiment of a method of automatedly ranking dermatological images; and

FIG. 5 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Embodiments of the disclosed systems and methods combine a platform for collecting dermatological images subject to set quality standards, and artificial intelligence and computer vision techniques to determine the quality level of a set of dermatological images, where the quality level is a reflection of the degree to which a given image depicts an anatomical feature, such as a lesion or abnormality of the skin, clearly. Dermatological image collection may involve capture of many images in rapid succession, at varying camera focus lengths, exposure times, and other hardware quality metrics. Computer vision techniques may be used to determine whether a set of images contains sufficiently good general quality and/or whether a portion of the image containing an anatomical feature of interest is of high quality. Multiple images may be ranked according to their quality as defined above, for categorization of data banks of images and/or selection of a superior sample image. Artificial intelligence may be used to identify an anatomical feature of interest, either by recognizing a most probably significant feature based on training sets illustrating various anatomical features, or by matching a user command indicating a category of anatomical feature to an image detail matching the category.

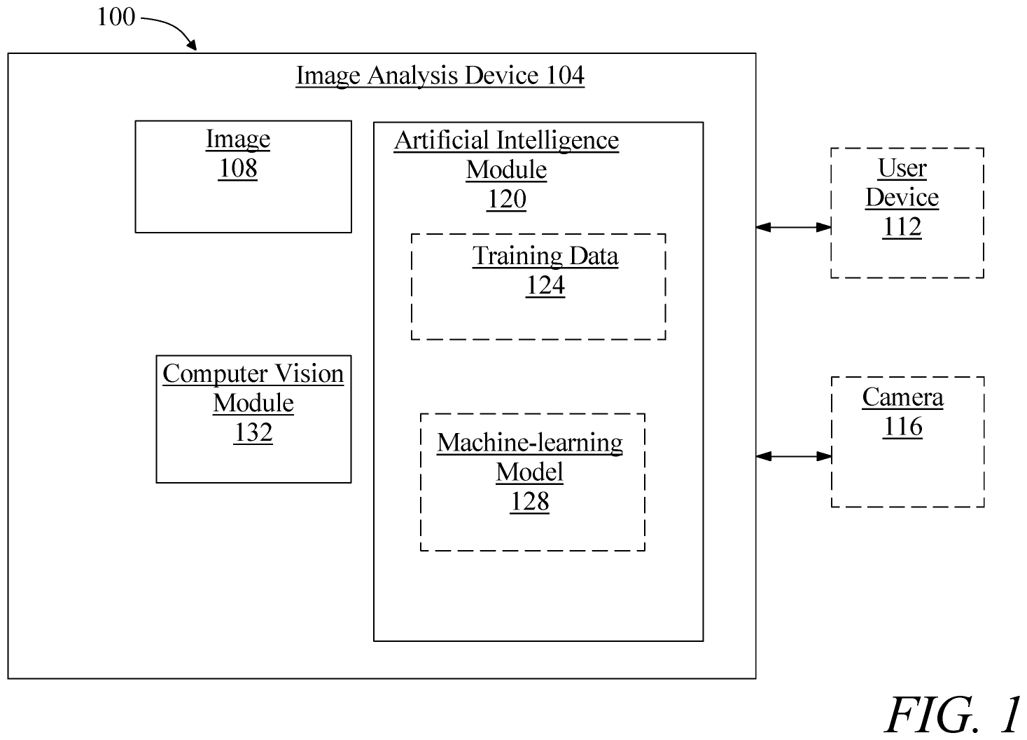

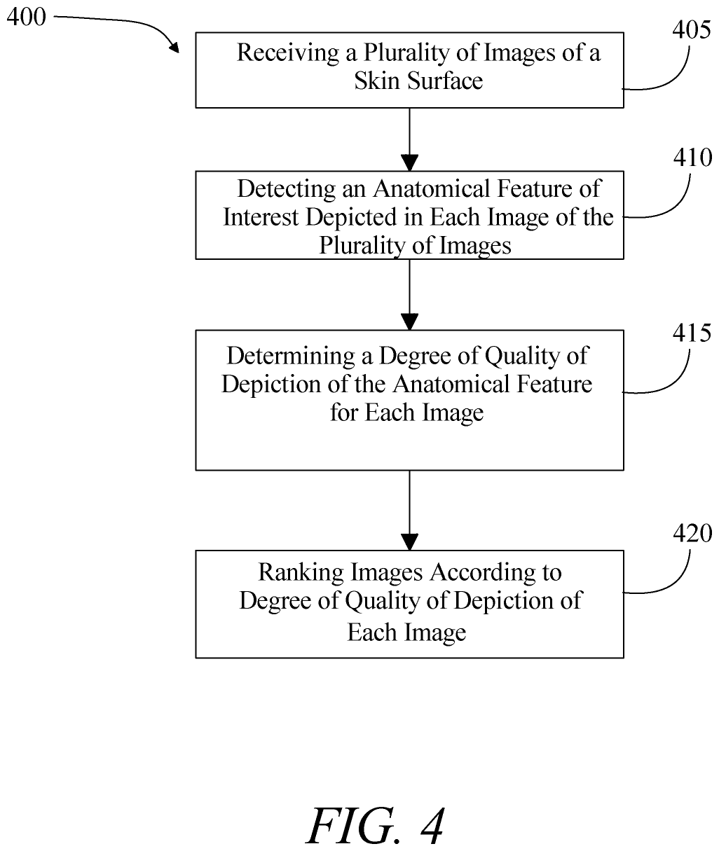

Referring now to FIG. 1, an exemplary embodiment of a system 100 for automatedly ranking dermatological images is illustrated. System 100 includes an image analysis device 104. Image analysis device 104 may include any computing device as described below in reference to FIG. 4, including without limitation a microcontroller, microprocessor, digital signal processor (DSP) and/or system on a chip (SoC) as described below in reference to FIG. 6. Image analysis device 104 may include, be included in, and/or communicate with a mobile device such as a mobile telephone or smartphone. Image analysis device 104 may include a single computing device operating independently, or may include two or more computing devices operating in concert, in parallel, sequentially or the like; two or more computing devices may be included together in a single computing device or in two or more computing devices. Image analysis device 104 with one or more additional devices as described below in further detail via a network interface device. Network interface device may be utilized for connecting an image analysis device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Image analysis device 104 may include but is not limited to, for example, an image analysis device 104 or cluster of computing devices in a first location and a second computing device or cluster of computing devices in a second location. Image analysis device 104 may include one or more computing devices dedicated to data storage, security, distribution of traffic for load balancing, and the like. Image analysis device 104 may distribute one or more computing tasks as described below across a plurality of computing devices of computing device, which may operate in parallel, in series, redundantly, or in any other manner used for distribution of tasks or memory between computing devices. Image analysis device 104 may be implemented using a “shared nothing” architecture in which data is cached at the worker, in an embodiment, this may enable scalability of system 100 and/or computing device.

Still referring to FIG. 1, image analysis device 104 may be designed and/or configured to perform any method, method step, or sequence of method steps in any embodiment described in this disclosure, in any order and with any degree of repetition. For instance, image analysis device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Image analysis device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

As a non-limiting example, and with continued reference to FIG. 1, image analysis device 104 may be designed and configured to receive at least an image 108 of a skin surface. At least an image 108 may include a plurality of images. At least an image 108 may include at least a digital image, either taken using a digital camera or converted to a digital image from a non-digital photographic form using, without limitation, a camera, scanner, or other optical device for image conversion and/or capture. Each image of at least an image 108 may depict at least an anatomical feature of interest. An “anatomical feature of interest,” as used herein, is a visible feature of a person's skin that the person may like to be viewed and/or evaluated by another person such as without limitation a doctor, dermatologist, nurse practitioner, or the like; an anatomical feature of interest may include a feature that a person reviewing at least an image 108, such as a doctor, dermatologist, nurse practitioner, or the like, may wish to view. Anatomical features may include, without limitation, anatomical features representing a state of cutaneous health and/or other skin condition of a user, including any form of lesions, moles, spots, warts, benign or malignant growths including skin cancer, “skin tags” or the like, cuts, bruises, hematomas, boils, abscesses, infections, parasitic infestations, ticks, animal bites, rashes, allergic reactions, local symptoms of psoriasis, abrasions, burns, dry areas, cracks, pimples, acne, or the like, and excluding biometric features such as fingerprints. Identification of an anatomical feature of interest as used herein is distinct from identification of a body part excepting a portion of skin and/or a feature thereof; for instance, and without limitation identification is a distinct process from facial recognition.

Still referring to FIG. 1, at least an image 108 may include a single image; alternatively or additionally, at least an image 108 may include multiple images. Each of multiple images may include an image of the same general portion of the same person's skin; for instance, an anatomical feature of interest depicted in each image of the plurality of images may be identical to the anatomical feature of interest depicted in each other image of the plurality of images. Alternatively or additionally, a plurality of images may depict various different samples, including images of different persons' skin, different portions of skin on a person, or the like. Various different samples may have one or more category in common; for instance, images may depict various samples having the same category of anatomical feature, various samples from a particular person, family, ethnic group, age group, sex, and/or other medical or demographic variable. Multiple images may include a plurality of images where each image of the plurality of images has at least an image capture parameter differing from an image capture parameter of each other image of the plurality of images; image capture parameter may include any parameter affecting circumstances and/or manner of image capture, including without limitation focal length, filter, lighting, aperture, film speed (digital or analog), frame rate, image resolution (e.g. in pixels), color filter (whether physical or virtual), wavelengths captured, or the like. For instance, and without limitation, some images of plurality of images may be taken with flashes, some without, some with wide apertures, some with narrow apertures, at varied angles, and/or at varied focal lengths.

Further referring to FIG. 1, a plurality of images may be taken as a “burst” of images by a camera, as a video feed including without limitation live-streamed video, or the like. A “burst” of images, as used in this disclosure, is a set of images of a single subject, such as a single area of skin or anatomical feature, taken in rapid succession. A burst may be performed by repeated manually actuated image captures, or may be an “automated burst,” defined as a set of images that are automatically triggered by a camera, computing device, image capture device, or the like; an automated burst may be initiated by a manual actuation of, e.g., a camera button while in an automated burst mode configuring an image capture device and/or computing device to perform and/or command an automated burst upon a manual actuation, or may be triggered by an automated process and/or module such as a program, hardware component, application, a command or instruction from a remote device, or the like. In an embodiment, image analysis device 104 and/or another computing device may automatically direct and/or generate a burst or sequence of images as described in further detail below. As a non-limiting example, image analysis device 104 may be configured to receive the plurality of images by generating a first image capture parameter, transmitting a command to a camera and/or user device to take at least a first image of the plurality of digital images with the first image capture parameter, generating a second image capture parameter, transmitting a command to the camera and/or user device to take at least a second image of the plurality of digital images with the second image capture parameter, and receiving, from the camera and/or user device, the at least a first image and the at least second image. “Transmitting,” as used herein, may include transmission of a command from a processor in image analysis device 104 to a component thereof such as an integrated or attached camera, as well as remote transmission via wired or wireless network.

With continued reference to FIG. 1, image analysis device 104 may receive at least image via any electronic communication; for instance, image analysis device 104 may receive at least an image 108 from a user device 112. User device 112 may include any computing device suitable for use as image analysis device 104, including without limitation a user mobile device; for instance, a user may capture at least an image 108 using a camera incorporated in and/or in communication with user device 112, and transmit the image to image analysis device 104. Image may be transmitted via any suitable electronic communication protocol, including without limitation packet-based protocols such as transfer control protocol-internet protocol (TCP-IP), file transfer protocol (FTP) or the like. Image may be transmitted via a text messaging service such as simple message service (SMS) or the like. Image may be received via a portable memory device such as a disc or “flash” drive, via local and/or near-field communication, or according to any other direct or indirect means for transmission and/or transfer of digital images. Receiving at least an image 108 may include retrieval of at least an image 108 from a database and/or datastore containing images; at least an image 108 may be retrieved using a query that, for instance, specifies a category as described above that one or more images may be required to match.

Still referring to FIG. 1, system 100 may include a camera 116, which may be used to capture at least an image 108. Camera 116 may include any digital camera 116 incorporated in or in communication with image analysis device 104. Camera 116 may incorporate or be incorporated in a computing device, which may include any computing device suitable for use as image analysis device 104 as described above. In an embodiment, receiving at least an image 108 may include capturing the at least an image 108 using camera 116. Camera 116 may be configured to take a plurality of digital images. Camera 116 may be designed and configured to receive a command to take at least a first image of the plurality of digital images with a first image capture parameter, which may include any image capture parameter as described above, and capture at least a first image of the plurality of digital images with the first capture parameter. Camera 116 may be configured to receive a command to take at least a second image of the plurality of digital images with a second image capture parameter, which may include any parameter suitable for use as a first image capture parameter and capture the at least a second image with the second image capture parameter. Second image capture parameter may differ from first image capture parameter. Camera 116 may be configured to receive a plurality of commands to capture a plurality of images, each image of the plurality of image having an image capture parameter differing from an image capture parameter of at least one other image of the plurality of images.

With continued reference to FIG. 1, image analysis device 104 may be configured to generate the first image capture parameter. Image analysis device 104 may, for instance, select first image capture parameter from a list and/or range of possible values of focal length, aperture, film speed, or the like; first parameter may be randomly selected from sequence or range, may be selected as an upper or lower extreme achievable by camera 116, such as without limitation, a maximal or minimal focal length, aperture, and/or film speed, a median, mean or other value, or any other suitable selection that may occur to a person skilled in the art upon reviewing the entirety of this disclosure. List or range may be a list or range of physically possible parameters for camera 116, a list or range of useful parameters, and/or a list or range of values selected by a user and/or stored as representing a range of parameters covering a desired degree of variation between pictures. Image analysis device 104 may be configured to transmit a command to the camera to take the at least a first image of the plurality of digital images with the first image capture parameter; this may be performed according to any method for transmission of a command from an image analysis device 104 to a camera. Image analysis device 104 may be configured to generate a second image capture parameter; this may be accomplished using any process suitable for generating a first image capture parameter, including randomly selecting a differing value from a range and/or list of values, selecting a value at an opposite end of a range or some increment along the range from first value, and/or selecting a differing value in a list in any order, by traversal, or the like. Image analysis device 104 may be configured to transmit a command to camera 116 to take at least a second image of the plurality of digital images with the second image capture parameter. Image analysis device 104 may be configured to receive at least a first image and at least second image from camera 116. Image analysis device 104 may repeat this with a plurality of image capture parameters and/or commands and may receive a plurality of distinct images in return; plurality of images may be any plurality of images as described in this disclosure.

With continued reference to FIG. 1, system 100 may include an artificial intelligence module 120 operating on the image analysis device 104. Artificial intelligence module 120 may include, without limitation, any software module, hardware module, or combination thereof. Artificial intelligence module 120 may be designed and configured to detect an anatomical feature of interest depicted in the at least an image 108 of the skin surface. In an embodiment, artificial intelligence module 120 may receive a command indicating a category of anatomical feature, for instance as described above, to detect; alternatively or additionally, artificial intelligence module 120 may detect one or more anatomical features in at least an image 108; where multiple anatomical features are detected, artificial intelligence module 120 may, for instance, present the multiple features to a user to select a feature of interest. Alternatively or additionally, where multiple features are detected, artificial intelligence module 120 may rank the multiple features by severity, acuteness, or the like; for instance, an apparently cancerous lesion may be ranked higher than an ingrown hair. Artificial intelligence module 120 may present plurality of identifications and/or ranking to a user, such as a person from whom images were captured, a health-care professional such as a dermatologist or the like, or any other user, to aid in selection of a feature of interest; alternatively or additionally, artificial intelligence module 120 may automatedly select a highest-ranking anatomical feature as anatomical feature of interest. Severity, acuteness, and/or other ranking criteria may be received by image analysis device 104 and/or artificial intelligence module 120 from one or more experts such as without limitation dermatologists and/or other health-care professionals.

Still referring to FIG. 1, artificial intelligence module 120 may be designed and configured to identify an anatomical feature as a function of training data 124 stored on or accessible to image analysis device 104. Training data 124, as used herein, is data containing correlations that a machine-learning process may use to model relationships between two or more categories of data elements. For instance, and without limitation, training data 124 may include a plurality of data entries, each entry representing a set of data elements that were recorded, received, and/or generated together; data elements may be correlated by shared existence in a given data entry, by proximity in a given data entry, or the like. Multiple data entries in training data 124 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 124 according to various correlations; correlations may indicate causative and/or predictive links between categories of data elements, which may be modeled as relationships such as mathematical relationships by machine-learning processes as described in further detail below. Training data 124 may be formatted and/or organized by categories of data elements, for instance by associating data elements with one or more descriptors corresponding to categories of data elements. As a non-limiting example, training data 124 may include data entered in standardized forms by persons or processes, such that entry of a given data element in a given field in a form may be mapped to one or more descriptors of categories. Elements in training data 124 may be linked to descriptors of categories by tags, tokens, or other data elements; for instance, and without limitation, training data 124 may be provided in fixed-length formats, formats linking positions of data to categories such as comma-separated value (CSV) formats and/or self-describing formats such as extensible markup language (XML), enabling processes or devices to detect categories of data.

Alternatively or additionally, and still referring to FIG. 1, training data 124 may include one or more elements that are not categorized; that is, training data 124 may not be formatted or contain descriptors for some elements of data. Machine-learning algorithms and/or other processes may sort training data 124 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below.

With continued reference to FIG. 1, image analysis device 104 may be configured to receive a training set including a plurality of data entries, each data entry of the training set including at least an image 108 of an anatomical feature and at least a correlated label describing at least an anatomical feature. Training set may be compiled, as a non-limiting example, by provision of images to one or more experts, such as dermatologists or the like, and receipt of one or more labels associated with each image from the one or more experts. One or more experts may, for instance, label a given anatomical feature as depicting a mole, a potentially pre-cancerous mole, a wart, or the like. Labels may be entered by experts in textual form or selected from one or more lists of pre-selected labels presented, as a non-limiting example with checkboxes or in drop-down list form. Experts may similarly be asked to rate quality of images to train system 100 to detect quality levels as described in further detail below.

Still referring to FIG. 1, training data 124 may include two or more sets of training data. For instance, training data 124 may include one or more sets of demographically linked training data, where “demographically linked training data,” as used in this disclosure, is training data in which each data entry contains an image from a person having a demographic trait common to all data entries in the demographically linked training data. Demographic trait may include, without limitation, age, sex, gender, ethnicity, geographical region of residence, geographical region of birth, skin tone, and/or any other demographic factor that may affect appearance of skin and/or anatomical features. For instance, a first set of demographically linked training data may include training data in which all images are of fair-skinned people, a second set of demographically linked training data may include training data in which all images are of people with moderately toned or light-brown skin, and/or a third set of demographically linked training data may include training data in which all images are of people with darker-toned and/or dark brown skin. As a further non-limiting example, a first set of demographically linked training data may contain images of people belonging to a first ethnic group having a first range of skin tones, while a second set of demographically linked training data may contain images of people belonging to a second ethnic group having a second range of skin tones. In an embodiment, creation and/or collection of sets of demographically linked training data may be driven by a feedback process; for instance, where methods of anatomical feature detection and/or image quality ranking are less accurate, for example as rated by expert users viewing samples of results of methods, feedback may be entered indicating greater or lesser accuracy, and machine-learning processes and/or users may identify one or more demographic features common to a set of less accurate results, resulting in automatic and/or user-driven generation of a set of demographically linked training data. As a non-limiting example, a classifier as described below may be used to classify elements of training data to one or more demographic traits, and a set of training data classified to a given trait may be used as a set of demographically linked training data. Alternatively or additionally, where users enter demographic information along with images used in training data, sets of training data having a given demographic trait in common may be retrieved and/or collected using a database query.

Continuing to refer to FIG. 1, training data 124 may include two or more sets of image quality-linked training data. “Image quality-linked” training data, as described in this disclosure, is training data in which each training data element has a degree of image quality, according to any measure of image quality, matching a degree of image quality of each other training data element, where matching may include exact matching, falling within a given range of an element which may be predefined, or the like. For example, a first set of image quality-linked training data may include images having no or extremely low blurriness, while a second set of image quality-linked training data. In an embodiment, sets of image quality-linked training data may be used to train image quality-linked machine-learning processes, models, and/or classifiers as described in further detail below.

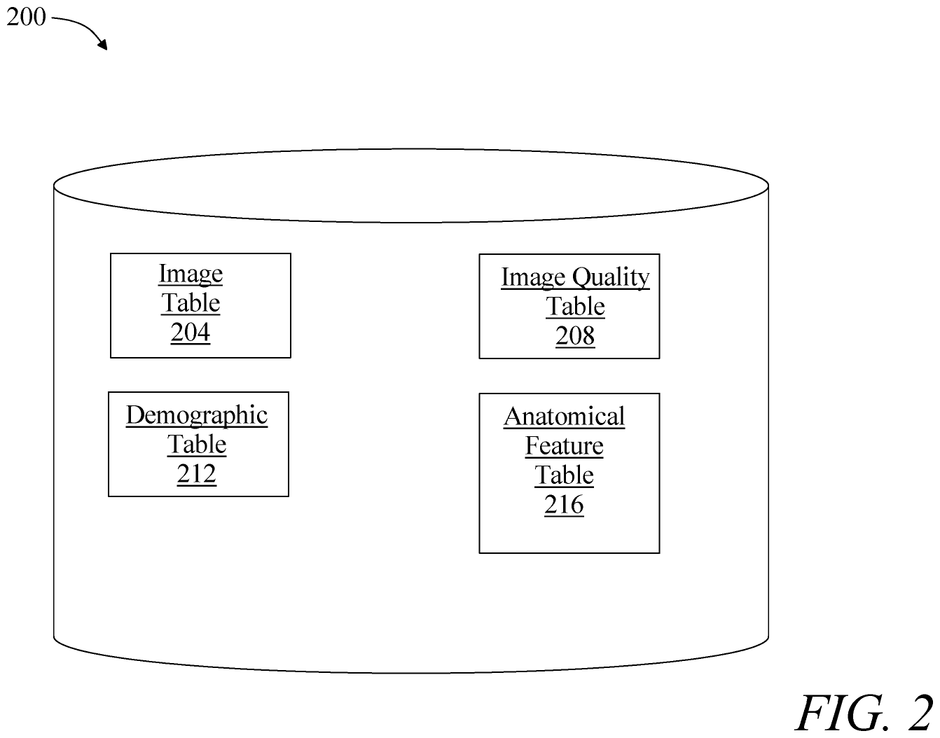

Referring now to FIG. 2, training data, images, and/or other elements of data suitable for inclusion in training data may be stored, without limitation, in an image database 200. Image database 200 may include any data structure for ordered storage and retrieval of data, which may be implemented as a hardware or software module. Image database 200 may be implemented, without limitation, as a relational database, a key-value retrieval datastore such as a NOSQL database, or any other format or structure for use as a datastore that a person skilled in the art would recognize as suitable upon review of the entirety of this disclosure. An image database 200 may include a plurality of data entries and/or records corresponding to user tests as described above. Data entries in an image database 200 may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in an image database 200 may reflect categories, cohorts, and/or populations of data consistently with this disclosure. Image database 200 may be located in memory of image analysis device 104 and/or on another device in and/or in communication with system 100.

Still referring to FIG. 2, an exemplary embodiment of an image database 200 is illustrated. One or more tables in image database 200 may include, without limitation, an image table 204, which may be used to store images, with links to origin points and/or other data stored in image database 200 and/or used in training data as described in this disclosure. Image database 200 may include an image quality table 208, where categorization of images according to image quality levels, for instance for purposes of use in image quality-linked training data, may be stored. Image database 200 may include a demographic table 212; demographic table may include any demographic information concerning users from which images were captured, including without limitation age, sex, national origin, ethnicity, language, religious affiliation, and/or any other demographic categories suitable for use in demographically linked training data as described in this disclosure. Image database 200 may include an anatomical feature table 216, which may store types of anatomical features, including links to diseases and/or conditions that such features represent, images in image table 204 that depict such features, severity levels, mortality and/or morbidity rates, and/or degrees of acuteness of associated diseases, or the like. Persons skilled in the art, upon reviewing the entirety of this disclosure, will be aware of various alternative or additional data which may be stored in image database 200.

Referring again to FIG. 1, artificial intelligence module 120 may be designed and configured to perform at least a machine-learning process to perform one or more determinations and/or other process steps described in this disclosure, including without limitation relation of images to anatomical features, classification of image data to demographic traits, image quality traits, and/or other traits and/or attributes, or the like. A “machine learning process,” as used in this disclosure, is a process that automatedly uses a body of data known as “training data” and/or a “training set” to generate an algorithm that will be performed by a computing device/module to produce outputs given data provided as inputs; this is in contrast to a non-machine learning software program where the commands to be executed are determined in advance by a user and written in a programming language.” For instance, and without limitation, image analysis device 104 may be configured create at least a machine-learning model 128 and/or enact a machine-learning process relating images of anatomical features to labels of anatomical features using the training set and generating the at least an output using the machine-learning model 128; at least a machine-learning model 128 may include one or more models that determine a mathematical relationship between images of anatomical features and labels of anatomical features. Such models may include without limitation model developed using linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 1, machine-learning algorithm used to generate machine-learning model 128 may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Still referring to FIG. 1, artificial intelligence module 120 may generate its output using alternatively or additional artificial intelligence methods, including without limitation by creating an artificial neural network, such as a convolutional neural network comprising an input layer of nodes, one or more intermediate layers, and an output layer of nodes. Connections between nodes may be created via the process of “training” the network, in which elements from a training data set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. This network may be trained using a training set; the trained network may then be used to apply detected relationships between elements of images of anatomical features and labels and or image quality of anatomical features.

Still referring to FIG. 1, machine-learning algorithms used by artificial intelligence module 120 may include supervised machine-learning algorithms. Supervised machine learning algorithms, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may use images of anatomical features as inputs, labels of anatomical features as outputs, and a scoring function representing a desired form of relationship to be detected between images of anatomical features and labels of anatomical features; scoring function may, for instance, seek to maximize the probability that a given element of images of anatomical features is associated with a given label of anatomical features to minimize the probability that a given element of images of anatomical features and/or combination of elements of images of anatomical features is not associated with a given label of anatomical features. Scoring function may be expressed as a risk function representing an “expected loss” of an algorithm relating inputs to outputs, where loss is computed as an error function representing a degree to which a prediction generated by the relation is incorrect when compared to a given input-output pair provided in training set. Persons skilled in the art, upon reviewing the entirety of this disclosure, will be aware of various possible variations of supervised machine learning algorithms that may be used to determine relation between images of anatomical features and labels of anatomical features. In an embodiment, one or more supervised machine-learning algorithms may be restricted to a particular domain; for instance, a supervised machine-learning process may be performed with respect to a given set of demographically linked training data 124, such as data showing images relating to a particular ethnic group, age group, sex, or the like.

With continued reference to FIG. 1, artificial intelligence module 120 may alternatively or additionally be configured to detect an anatomical feature of interest by executing a lazy learning process as a function of the training set and at least an image; lazy learning processes may be performed by a lazy learning module 308 executing on classification device 104 and/or on another computing device in communication with classification device 104, which may include any hardware or software module. A lazy-learning process and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce an on demand. For instance, an initial set of simulations may be performed to cover a “first guess” at an anatomical feature label associated with an image of an anatomical feature, using training set. As a non-limiting example, an initial heuristic may include a ranking of labels of anatomical features according to relation to a category of images of anatomical features or the like. Heuristic may include selecting some number of highest-ranking associations and/or labels of anatomical features. Artificial intelligence module 120 may alternatively or additionally implement any suitable “lazy learning” algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate labels of anatomical features as described in this disclosure, including without limitation lazy learning applications of machine-learning algorithms as described above.

Still referring to FIG. 1, machine learning processes may include unsupervised processes. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 1, machine-learning processes as described in this disclosure may be used to generate machine-learning models. A machine-learning model, as used herein, is a mathematical representation of a relationship between inputs and outputs, as generated using any machine-learning process including without limitation any process as described above, and stored in memory; an input is submitted to a machine-learning model once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model may be generated by creating an artificial neural network, such as a convolutional neural network comprising an input layer of nodes, one or more intermediate layers, and an output layer of nodes. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

With continued reference to FIG. 1, machine-learning processes may include a classification process and/or algorithm, including without limitation an algorithm for generating a classifier. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Image analysis device 104 and/or another device may generate a classifier using a classification algorithm, defined as a processes whereby an image analysis device 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, image analysis device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Image analysis device 104 may then calculate a likelihood table by calculating probabilities of different data entries and classification labels. Image analysis device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, image analysis device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 1, and as a non-limiting example, an image analysis device 104 and/or a component thereof may generate a classifier that classifies images to anatomical features; classifier may classify each image to one or more anatomical features. For instance, classifier may identify an anatomical feature most closely related to image based on, e.g., a measure of distance as described above in KNN processes, and/or all anatomical features within a threshold distance of the image. Alternatively or additionally, each image may be divided into two or more sections, and each section may be classified to an anatomical feature; sections may be generated by a regular randomized tessellation of the image, for instance into rectangles, triangles, and/or hexagons or may be selected by an initial process identifying one or more high-contrast regions and/or other regions of interest in the image. Alternatively or additionally, a user input may highlight and/or otherwise indicate an area of an image depicting an anatomical feature of concern to a user. Where classifier and/or other processes produce multiple anatomical features, image analysis device 104 may be configured to select a feature having a highest degree of severity, acuteness, or the like as described below.

Still referring to FIG. 1, image analysis device 104 and/or one or more modules and/or components thereof may be configured to generate one or more category-specific machine-learning models and/or classifiers, which may be generated using any category-specific training data as described above. For instance, and without limitation, any demographically linked training data as described above may be used to generate one or more demographically linked machine-learning models and/or classifiers, and/or to perform demographically linked machine-learning and/or classification processes. As a further non-limiting example, any image quality-linked training data as described above may be used to generate one or more image quality-linked machine-learning models and/or classifiers, and/or to perform image quality-linked machine-learning and/or classification processes. In an embodiment, an image analysis device 104 and/or a component and/or module thereof may generate a first classifier using image quality-linked training data containing high-quality images, selected for instance using ranking processes and/or image-quality assessment as described below, where the first classifier accepts high-quality images as inputs and outputs identifications of anatomical features and/or categories of anatomical features, and then using a second machine-learning and/or classification process to train the first classifier using image quality-linked training having images of lower quality, to produce a second classifier that accepts lower-quality images as input and outputs identifications of anatomical features and/or categories of anatomical features.

With continued reference to FIG. 1, image analysis device 104 and/or one or more modules and/or components thereof may be configured to generate one or more machine-learning models and/or classifiers, and/or to perform machine-learning and/or classification processes, to link one or more anatomical features to categories of anatomical features as described above.

Further referring to FIG. 1, image analysis device 104 may alternatively or additionally generate machine-learning processes, classification processes, machine-learning models and/or classifiers to classify training data entries to one or more categories, which may include any categories as described above; for instance, processes, models, and/or classifiers may classify elements of training data to demographic traits as described above, levels of image quality as described above, or the like. Classifications to categories may be used to sort training data into category-specific training data sets as described above, including without limitation demographically linked training data sets and/or image quality-linked training data sets as described above.

With continued reference to FIG. 1, image analysis device 104 and/or machine learning module 104 may perform one or more optimizations to enable live and/or real-time response to images being captured in a video feed such as a live stream. Optimizations may include reducing image resolution of at least an image to be captured; any machine-learning algorithm as described above may be performed using reduced-resolution versions of images in training sets, for instance and without limitation generating models and/or heuristics based on detection of anatomical features and/or image quality in reduced sets. An error function may be used to compare predictive/detection skill of low-resolution models to predictive/detection skill of high-resolution models; a target error range may be established, and a minimum resolution may be selected based on a target error range/tolerance. Machine-learning algorithms may be selected for models and/or heuristics amenable to rapid processing, such as linear function-based heuristics, and/or heuristics relating fewer inputs or categories of inputs to outputs. A cost function minimizing processing time while targeting a specified error tolerance as compared to optimally accurate models may be performed to select higher-speed algorithms, models, and/or heuristics having good quality or acceptable quality results, where a “loss function” is an expression of an output of which an optimization algorithm minimizes to generate an optimal result. Expression may be a linear combination of factors such as average error, average computational cycles, worst-case computational cycles, or the like, which may be selected according to a set of priorities provided by users and/or generated by a machine-learning process such as a linear regression algorithm. In an embodiment, high-speed methods may be used to select a plurality of candidates from a video stream, which may then be processed using lower speed “burst” processes after initial communication to a user.

Further referring to FIG. 1, image analysis device 104 may output identification of anatomical feature; for instance, image analysis device 104 may display identification on a display connected to and/or in communication with image analysis device 104 and/or may cause a user device or other remote device to display identification. Display of identification may include visual display and/or display and/or output in any other user-comprehensible form including audio output, tactile output, or the like. Display may be provided in conjunction with display of an image of plurality of images, including without limitation an image ranked and/or selected as having a highest degree of image quality as described in further detail below. Alternatively or additionally, identification may be transmitted to one or more devices and/or components; for instance identification may be transmitted, together with one or more images selected and/or ranked as having a highest and/or higher level of image quality, to a device, component, and/or module operating a repository and/or data store of images, permitting images to be stored therein along with identifications of anatomical features. Storage may be combined with any demographic data as described above, or any other data useful and/or suitable for identifying one or more categories of an image as described above. Storage facility may include without limitation an image database 200 as described above. Images, demographic data, other category data, and/or anatomical feature data collected and/or determined as described herein may in turn be used as training data for subsequent iterations of methods described herein.

Still referring to FIG. 1, system 100 may include a computer-vision module 132 operating on the image analysis device 104. Computer-vision module 132 may include any software module, hardware module, or combination thereof. Computer-vision module 132 is designed and configured to determine a degree of quality of depiction of the anatomical feature in the at least an image 108. Degree of quality of an image, as used herein, is the degree to which the image clearly depicts an identified anatomical feature of interest. In an embodiment, computer-vision module 132 may determine a degree of blurriness of an image. Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a Gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

In an embodiment, and still referring to FIG. 1, computer-vision module 132 may determine whether an image of at least an image 108 has anatomical feature of interest as a focal point. This may be determined by analyzing a degree of focus at a portion of an image containing anatomical feature of interest; this may be accomplished using any algorithm and/or operator as described above for blurriness detection and/or determination of degree of focus. Alternatively or additionally, computer-vision module 132 may perform a whole-image blurriness detection process with regard to a section of image containing anatomical feature of interest, including without limitation a section of image mostly or substantially filled by anatomical feature of interest. Computer-vision module 132 may determine a quality level according to a degree of lightness, darkness, contrast, or another parameter. The computer-vision module may also analyze a series of dermatological images taken in rapid succession of the same subject matter, but at varying camera lens focal lengths, exposure times, or the like. In this case the computer-vision module may identify the most in-focus image corresponding to a selected region of the image.

Continuing to view FIG. 1, computer-vision module 132 may also use artificial intelligence to determine a level of quality of an image; for instance, and without limitation, a training set may be collected, for instance from dermatologists or other experts as described above, where images are related to quality levels as determined by the dermatologists. A supervised machine-learning process may then be performed to teach computer-vision module 132 one or more mathematical formulas to determine a quality level of an image or identify locations in the image corresponding to skin or afflicted regions on the skin. In an embodiment, and without limitation, a classifier and/or classification process as described above may use training data to classify each image to an image quality level and/or category. Alternatively or additionally, image analysis device 104 may perform meta-analysis of machine-learning processes as described above; for instance, image analysis device may determine a degree to which machine-learning processes, classification processes, classifiers, and/or models for identifying anatomical features are able to identify an anatomical feature in each image. If identification of an anatomical feature in a first image is less certain than identification of an anatomical feature in a second image, where certainty is measured, e.g., by a degree to which classification produces an unambiguous and/or exclusive result and/or a minimal distance under distance metrics in KNN for instance from image to an identification of an anatomical feature, then the second image may be identified as higher quality than the first image.

Still referring to FIG. 1, in an embodiment image analysis device 104 may be configured to rank multiple images of at least an image 108 according to quality level, as determined by computer-vision module 132. A subset of at least an image 108 may be selected based on ranking; for instance only the highest-ranking image may be selected, or a certain number of highest-ranking images, while non-selected images are discarded. Alternatively or additionally, any image of at least an image 108 having less than a threshold level of quality may be discarded; where no image meets the threshold level, a request may be displayed and/or transmitted to a user, for instance via user device 112, for one or more additional images to be provided. For instance, and without limitation, image analysis device 104 may be configured to select at least a highest-ranking image of the plurality of images and transmit the at least a highest-ranking image to a remote device. As a further example, image analysis device 104 may be configured to select at least a highest-ranking image of the plurality of images and transmit the at least a highest-ranking image to an image repository, such as without limitation image database 200 as described above; highest-ranking images may be transmitted thereto and/or stored therein along with any identifications of anatomical features, demographic traits, and/or other categories as described above. In an embodiment, where no image or too few images fall below a predetermined and/or stored threshold of quality, image analysis device and/or a component and/or module thereof may prompt a user to obtain more images and/or automatically generate a command to take more images as described in this disclosure; this may be repeated iteratively until one or more images of sufficient quality are obtained.

In an embodiment, and with continued reference to FIG. 1, a plurality of images may be selected by any version of any method step as described herein and presented to user device 112. A user such as a dermatologist and/or other professional may select a preferred image or images and enter the selection at user device 112; this may cause user device 112 to display the selected image or images. Selection may be entered as an additional value in training data 124 as described above

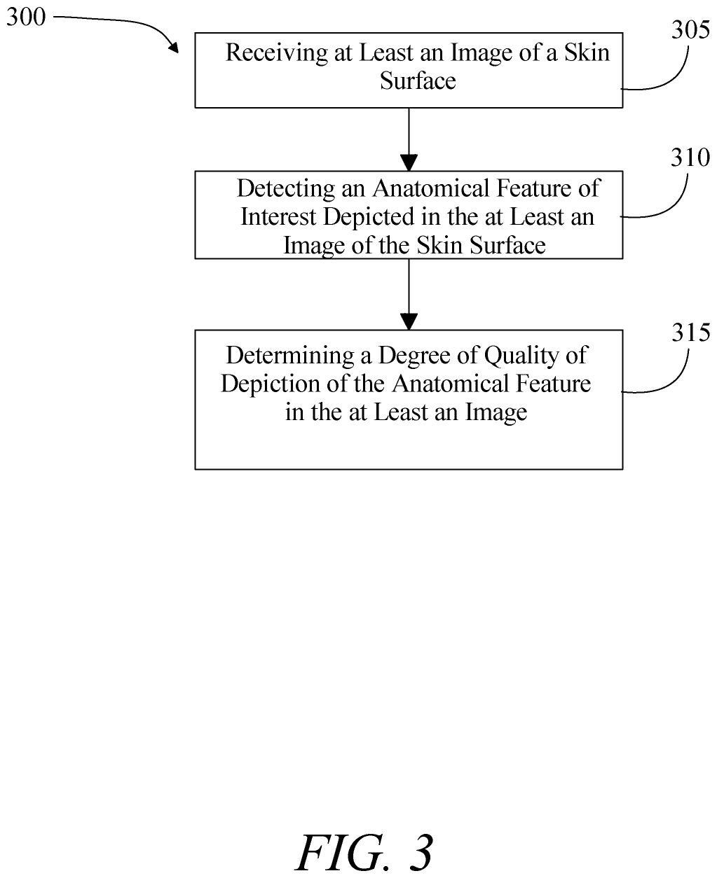

Referring now to FIG. 3, an exemplary embodiment of a method 300 of automatedly evaluating dermatological images is illustrated. At step 305, an image analysis device 104 image analysis device 104 receives at least an image 108 of a skin surface; this may be implemented, for instance, as described above. In an embodiment, the at least an image 108 may be retrieved from a database or data store of such images, such as are kept by institutions, companies, medical facilities, or the like; at least an image 108 alternatively be received from a user device 112. In an embodiment, image analysis device 104 iterates through stored image data to classify it or determine quality levels. At a step 310, an artificial intelligence module 120 operating on the image analysis device 104 detects an anatomical feature of interest depicted in the at least an image 108 of the skin surface. This may be implemented, without limitation, as described above in reference to FIG. 1. In an embodiment, image analysis device 104 appends one or more labels indicating anatomical features of interest and/or categories thereof to a data record containing or associated with the at least an image 108; method 300 may, for instance, iteratively label all images with all skin conditions, abnormalities, or the like represented. In an embodiment, method 300 may further include evaluating a degree of alleviation of a symptom or condition in a temporally sequential set of images provided as the at least an image 108.

At step 315, and still referring to FIG. 3, a computer-vision module 132 operating on the image analysis device 104 determines a degree of quality of depiction of the anatomical feature in the at least an image 108; this may be implemented as described above in reference to FIG. 1. Step 315 may be performed before, after, or in part before and in part after step 310; for instance, an overall assessment of blurriness may be performed on images, and images that are determined to be overly blurry may be discarded, prior to any attempt to identify anatomical features within images. Images having quality below a threshold may be deleted from a database or data store of images, and/or may be flagged as poor quality for review by a user who may decide whether to delete them.

Referring now to FIG. 4, an exemplary embodiment of a method 400 of automatedly ranking dermatological images is illustrated. At step 405, an image analysis device 104 receives a plurality of images of a skin surface; this may be implemented for instance as described above in reference to FIG. 1. Each image of the plurality of images may have at least an image capture parameter differing from an image capture parameter of each other image of the plurality of images; for instance, and as described in further detail above, each image may have a different focal length, lighting level, aperture size, film speed, or the like. Each image may an anatomical feature identical to an anatomical feature of interest depicted in each other image of the plurality of images; for instance, a user may take a series or “burst” of images all of the same spot or area of the user's skin, with differing image capture parameters, for example with a purpose of selecting an optimal image to send to a dermatologist or the like for a diagnosis or treatment recommendation.

At step 410, and still referring to FIG. 4, an artificial intelligence module 120 operating on the image analysis device 104 detects an anatomical feature of interest depicted in each image of the plurality of images; this may be performed, for instance, as described above in reference to FIG. 1. In an embodiment, a user may enter a command indicating a category of anatomical feature for artificial intelligence module 120 to search for; alternatively or additionally, artificial intelligence module 120 may detect one or more features consistent with one or more conditions, and system 100 may display labels, descriptions, or the like associated with such features to user, for instance as an initial warning system or “pre-diagnosis.”

At step 415, and continuing to refer to FIG. 4, a computer-vision module 132 operating on the image analysis device 104 determines a degree of quality of depiction of the anatomical feature for each image; this may be performed as described above in reference to FIG. 1. At step 420, image analysis device 104 ranks images according to degree of quality of depiction of the image. This may be implemented, without limitation, as described above in reference to FIG. 1. A highest-ranking image may be displayed and/or transmitted to the user, an expert, a dermatologist, or the like.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

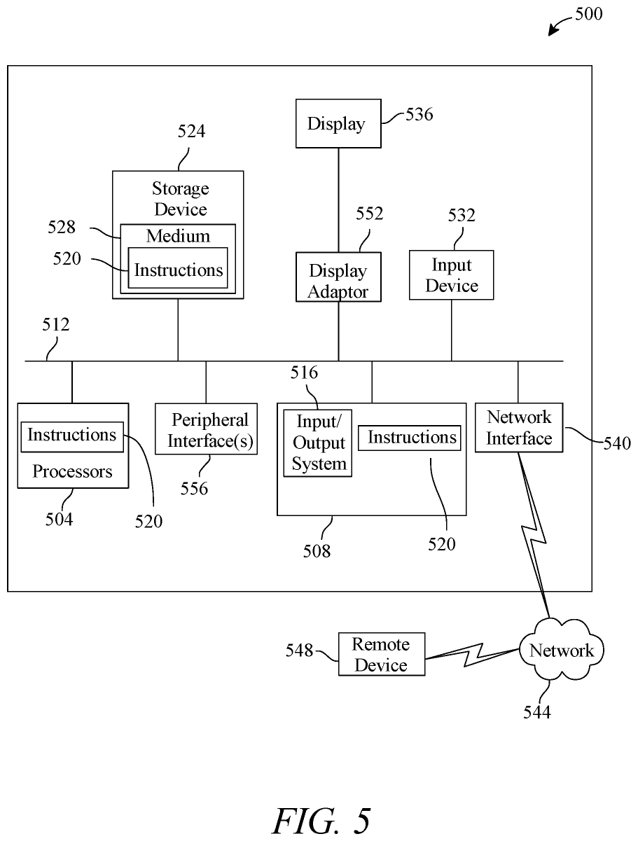

FIG. 5 shows a diagrammatic representation of one embodiment of a computing device in the exemplary form of a computer system 500 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 500 includes a processor 504 and a memory 508 that communicate with each other, and with other components, via a bus 512. Bus 512 may include any of several types of bus structures including, but not limited to, a memory bus, a memory controller, a peripheral bus, a local bus, and any combinations thereof, using any of a variety of bus architectures.

Processor 504 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 504 may be organized according to Von Neumann and/or Harvard architecture as a non-limiting example. Processor 504 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC)

Memory 508 may include various components (e.g., machine-readable media) including, but not limited to, a random-access memory component, a read only component, and any combinations thereof. In one example, a basic input/output system 516 (BIOS), including basic routines that help to transfer information between elements within computer system 500, such as during start-up, may be stored in memory 508. Memory 508 may also include (e.g., stored on one or more machine-readable media) instructions (e.g., software) 520 embodying any one or more of the aspects and/or methodologies of the present disclosure. In another example, memory 508 may further include any number of program modules including, but not limited to, an operating system, one or more application programs, other program modules, program data, and any combinations thereof.

Computer system 500 may also include a storage device 524. Examples of a storage device (e.g., storage device 524) include, but are not limited to, a hard disk drive, a magnetic disk drive, an optical disc drive in combination with an optical medium, a solid-state memory device, and any combinations thereof. Storage device 524 may be connected to bus 512 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 524 (or one or more components thereof) may be removably interfaced with computer system 500 (e.g., via an external port connector (not shown)). Particularly, storage device 524 and an associated machine-readable medium 528 may provide nonvolatile and/or volatile storage of machine-readable instructions, data structures, program modules, and/or other data for computer system 500. In one example, software 520 may reside, completely or partially, within machine-readable medium 528. In another example, software 520 may reside, completely or partially, within processor 504.

Computer system 500 may also include an input device 532. In one example, a user of computer system 500 may enter commands and/or other information into computer system 500 via input device 532. Examples of an input device 532 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 532 may be interfaced to bus 512 via any of a variety of interfaces (not shown) including, but not limited to, a serial interface, a parallel interface, a game port, a USB interface, a FIREWIRE interface, a direct interface to bus 512, and any combinations thereof. Input device 532 may include a touch screen interface that may be a part of or separate from display 536, discussed further below. Input device 532 may be utilized as a user selection device for selecting one or more graphical representations in a graphical interface as described above.

A user may also input commands and/or other information to computer system 500 via storage device 524 (e.g., a removable disk drive, a flash drive, etc.) and/or network interface device 540. A network interface device, such as network interface device 540, may be utilized for connecting computer system 500 to one or more of a variety of networks, such as network 544, and one or more remote devices 548 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 544, may employ a wired and/or a wireless mode of communication. In general, any network topology may be used. Information (e.g., data, software 520, etc.) may be communicated to and/or from computer system 500 via network interface device 540.

Computer system 500 may further include a video display adapter 552 for communicating a displayable image to a display device, such as display device 536. Examples of a display device include, but are not limited to, a liquid crystal display (LCD), a cathode ray tube (CRT), a plasma display, a light emitting diode (LED) display, and any combinations thereof. Display adapter 552 and display device 536 may be utilized in combination with processor 504 to provide graphical representations of aspects of the present disclosure. In addition to a display device, computer system 500 may include one or more other peripheral output devices including, but not limited to, an audio speaker, a printer, and any combinations thereof. Such peripheral output devices may be connected to bus 512 via a peripheral interface 556. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for automatedly ranking dermatological images, the system comprising an image analysis device, the image analysis device designed and configured to:
receive a plurality of images of a skin surface;
detect, using a machine-learning process, an anatomical feature of interest in each image of the images of the skin surface;
determine a degree of quality of depiction of the anatomical feature in each image of the plurality of images; and
rank the plurality images according to degree of quality of depiction of the anatomical feature in each image.

2. The system of claim 1, wherein each image of the plurality of images has at least an image capture parameter differing from an image capture parameter of each other image of the plurality of images.

3. The system of claim 1, wherein the plurality of images further comprises a burst of images of an area of skin.

4. The system of claim 1, wherein the image analysis device is further configured to receive the plurality of images by:
generating a first image capture parameter;
transmitting a command to a camera to take at least a first image of the plurality of digital images with the first image capture parameter;
generating a second image capture parameter;
transmitting a command to the camera to take at least a second image of the plurality of digital images with the second image capture parameter; and
receiving, from the camera, the at least a first image and the at least second image.

5. The system of claim 1, wherein the anatomical feature of interest depicted in each image of the plurality of images is identical to the anatomical feature of interest depicted in each other image of the plurality of images.

6. The system of claim 1, wherein the image analysis device is further configured to detect the anatomical feature of interest by:
detecting a plurality of anatomical features;
ranking the plurality of anatomical features by severity; and
selecting a highest-ranking anatomical feature of the plurality of anatomical features.

7. The system of claim 1, wherein:
the machine-learning process includes a machine-learning process using demographically linked training data; and
the image analysis device is further configured to match the plurality of images to the demographically linked training data.

8. The system of claim 1, wherein the machine-learning process includes a machine-learning process classifying the plurality of images to a category of anatomical feature

9. The system of claim 1, wherein the image analysis device is further configured to determine the degree of quality of depiction by determining a degree of blurriness of each image.

10. The system of claim 1, wherein the image analysis device is further configured to determine the degree of quality of depiction by determining a degree of focus at a portion of each image containing the anatomical feature of interest.

11. A method of automatedly ranking dermatological images, the method comprising:
receiving, by an image analysis device, a plurality of images of a skin surface;
detecting, by the image analysis device and using a machine-learning process, an anatomical feature of interest in each image of the images of the skin surface;
determining, by the image analysis device, a degree of quality of depiction of the anatomical feature in each image of the plurality of images; and
ranking, by the image analysis device, the plurality images according to degree of quality of depiction of the anatomical feature in each image.

12. The method of claim 14, wherein each image of the plurality of images has at least an image capture parameter differing from an image capture parameter of each other image of the plurality of images.

13. The method of claim 14, wherein the plurality of images further comprises a burst of images of an area of skin.

14. The method of claim 14, wherein receiving the plurality of images further comprises:
generating a first image capture parameter;
transmitting a command to a camera to take at least a first image of the plurality of digital images with the first image capture parameter;
generating a second image capture parameter;
transmitting a command to the camera to take at least a second image of the plurality of digital images with the second image capture parameter; and
receiving, from the camera, the at least a first image and the at least second image.

15. The method of claim 14, wherein the anatomical feature of interest depicted in each image of the plurality of images is identical to the anatomical feature of interest depicted in each other image of the plurality of images.

16. The method of claim 14, wherein detecting the anatomical feature of interest further comprises:
detecting a plurality of anatomical features;
ranking the plurality of anatomical features by severity; and
selecting a highest-ranking anatomical feature of the plurality of anatomical features.

17. The method of claim 14, wherein the machine-learning process includes a machine-learning process using demographically linked training data, and further comprising matching the plurality of images to the demographically linked training data.

18. The method of claim 14, wherein the machine-learning process includes a machine-learning process classifying the plurality of images to a category of anatomical feature

19. The method of claim 14, wherein determining the degree of quality of depiction further comprises determining a degree of blurriness of each image.

20. The method of claim 14, wherein determining the degree of quality of depiction further comprises determining a degree of focus at a portion of each image containing the anatomical feature of interest.
